(19) United States
(12) Patent Application Publication
(10) Pub. No.: US 2026/0130849 A1
(43) Pub. Date: May 14, 2026

(54) Edaravone Oral Solution Compositions

(71) Applicant: Tulex Pharmaceuticals, Inc., Cranbury, NJ (US)

(72) Inventors: Yu-Hsing Tu, West Windsor, NJ (US); Yingjun Fan, Cranbury, NJ (US); Ashok Perumal, Monmouth Junction, NJ (US); James A. Lee, West Windsor, NJ (US); Shanmuka Harish Chalamuri, Plainsboro, NJ (US)

(21) Appl. No.: 19/386,502

(22) Filed: Nov. 12, 2025

Related U.S. Application Data
(60) Provisional application No. 63/719,981, filed on Nov. 13, 2024.

(51) Int. Cl. A61K 9/08; A61K 9/00; A61K 31/4152; A61K 47/02; A61K 47/10; A61K 47/20

(52) U.S. Cl. CPC A61K 9/08; A61K 9/0053; A61K 31/4152; A61K 47/02; A61K 47/10; A61K 47/20

(57) ABSTRACT

The invention relates to an oral solution of edaravone of purified water, sodium bisulfite, L-Cysteine hydrochloride monohydrate, propylene glycol and edaravone. The edaravone is present in the oral solution at a concentration of about 76 mg/5 mL. The oral solution may be administered for treating ALS.

### BACKGROUND OF THE INVENTION

U.S. Pat. No. 10,987,341 is directed to edaravone suspensions for oral administration. The suspensions generally include edaravone particles, a dispersant and water. The product can be in the form of a kit with a solid composition of edaravone particles and a dispersant solution. The two parts are mixed together to form a suspension that is administered to the patient. As examples of dispersants, the '341 patent lists polyvinyl alcohol, sucrose fatty acid ester, polysorbate, methylcellulose and hypromellose. A thickening agent may be used so that the edaravone particles maintain a well dispersed state for a long period of time. As examples of thickening agents, the '341 patent lists carmellose sodium, dextrin, tragacanth powder and xanthan gum. The '341 patent further describes using a sweetener to adjust the taste of the suspension and delay the settling of the edaravone particles by increasing the solution density of the suspension, with sugar being listed as a suitable sweetener that increases the density.

The '341 patent also notes that edaravone has the property of being susceptible to oxidation by dissolved oxygen in a liquid. As an example of a stabilizer against oxidation, the '341 patent lists antioxidants such as polyethylene glycol. The antioxidant should be present in an amount of 0.001-0.5% (w/v). The '341 patent lists as a liquid medium, termed a dispersion medium, that may be used in a suspension as being water and may contain an organic solvent such as propylene glycol, polyethylene glycol and ethanol. While the '341 patent does not provide the amount of propylene glycol, polyethylene glycol, or ethanol to be used in the aqueous dispersion medium, the amount should be used with the water in an amount to provide a suspension product.

The '341 patent discloses the edaravone compositions as including a dispersant, such as polyvinyl alcohol, methylcellulose, hypromellose, sucrose fatty acid ester, and polysorbate. The dispersant functions to disperse the edaravone particles in water such that the dispersant maintains the edaravone particles in a solid particle state in the water.

The '341 patent is listed in the U.S. Food and Drug Administration's Orange Book for the RADICAVA ORS® (105 mg/5 mL) oral suspension drug product. RADICAVA ORS® is indicated for the treatment of amyotrophic lateral sclerosis (ALS). The prescribing information for RADICAVA ORS® states that the suspension is supplied in 60 mL amber glass bottles. The product contains as inactive ingredients: L-cysteine hydrochloride hydrate, polyvinyl alcohol, simethicone emulsion, sodium bisulfite, sorbitol, and xanthan gum with phosphoric acid and sodium hydroxide added to adjust to pH 4. According to the storage and handling instructions for pharmacies, the product must be stored upright at refrigerated conditions of 2° C. to 8° C., without freezing. The product must be protected from light. According to the storage and handling instructions for patients, the product must be stored upright at room temperature between 20° C. to 25° C. and protected from light. The patient product must be discarded 15 days after opening the bottle or, if unopened, 30 days from the date of shipment indicated on the carton pharmacy label.

RADICAVA® injection is a clear, colorless liquid containing 30 mg of edaravone in 100 mL of isotonic, sterile, aqueous solution. The solution also contains L-cysteine hydrochloride hydrate and sodium bisulfite. Sodium chloride is added for isotonicity and phosphoric acid and sodium hydroxide are added to adjust to pH 4. The injection product is supplied in a polypropylene bag that is further overwrapped with polyvinyl alcohol secondary packaging. The overwrapped package contains an oxygen absorber and an oxygen indicator to minimize oxidation. According to the prescribing information for RADICAVA®, the product should not be used if the indicator has changed color before opening the packaging and once the overwrap package is opened, it should be used within 24 hours. The prescribing information also instructs that the drug product be inspected visually for particulate matter and discoloration prior to administration. It is evident that the currently marketed injectable solution of edaravone has limited stability and issues with particulate formation.

The inventors have determined that the currently available orally administered edaravone suspension has limitations related to patient convenience and product stability. Further, the inventors have recognized that because the orally administered product is in the form of a suspension, there can be concerns about settling of the drug particles over time. Even if the product is shaken in an attempt to disperse the settled particles, there is no certainty that the drug is homogenously dispersed. As a consequence, a patient administered the suspension may undesirably not receive an accurate dosing.

### SUMMARY OF THE INVENTION

The edaravone formulations of the invention can be formulated as edaravone oral solutions that are stable and ready to use. The edaravone oral solutions can be formulated in concentration ranges of 1 mg/mL to 50 mg/mL.

Edaravone has very low solubility and is chemically unstable in purified water. The invention relates to a solvent/co-solvent system that achieve a stable drug product in which the edaravone is in solution. The inventors determined that edaravone can be soluble in a solvent or solubilizing enhancing agent, for example propylene glycol, and polyethylene glycol 400. In one embodiment, the edaravone oral solution can be formulated in a concentration of 21 mg/ml in a co-solvent system containing propylene glycol and water as solvents with a maximum water content up to 30% (w/w). In another embodiment, the pH of the solution is 4.3 and the density is 1.05 mg/ml.

In one specific embodiment, the edaravone oral solution may be formulated at a concentration of 105 mg edaravone in 5 mL in co-solvent system comprising propylene glycol (83% w/v), purified water (20% w/v), sodium bisulfite (0.05% w/v), and L-Cysteine hydrochloride hydrate (0.025% w/v). This concentration was found to be stable for 3 months at 25° C./60% RH as well as at 2 to 8° C.

In one embodiment the edaravone oral solution uses propylene glycol and water with the composition having water at up to about 30% w/w and at up to about 20% w/w. For example, an edaravone oral solution with propylene glycol/water can have a strength of about 76 mg/5 mL with a propylene glycol/water ratio of about 70/30 to about 99/1 (i.e., up to about 30% w/w water) or a strength of about 105/mg/5 mL with a propylene glycol/water ratio of about 80/20 to about 99/1 (i.e., up to about 20% w/w water). In another embodiment, the edaravone oral solution has a propylene glycol at 80% (w/v) and water at 22% (w/v) to give a propylene glycol/water ratio of about 3.6/1.

It is an object of the invention to provide an edaravone oral solution composition in which the edaravone active ingredient is completely dissolved or essentially completely dissolved. Therefore there would be no particles of edaravone present or substantially no particles of edaravone present in the composition.

It is an objective of the invention for the edaravone oral solution composition to be stable for three month, six months, one year or two years when stored at room temperature. In other embodiments it is an objective of the invention for the edaravone oral solution composition to be stable for 6 months when stored under refrigerated conditions at 2-8° C. and when stored at 25° C./60% RH. In other embodiments it is an objective of the invention for the edaravone oral solution composition to be stable for up to 6 months at 30° C./65% RH and/or at 40° C./75% RH for up to 2 months. In one embodiment the composition has a dose of about 66-86 mg edaravone in solution.

In one embodiment the solution composition of 66-86 mg edaravone is bioequivalent to the RADICACA ORS® edaravone suspension composition containing 105 mg edaravone.

In one embodiment the solution is stable for at least three month to six months and potentially one year when stored at room temperature. In another embodiment, the solution is stable for at least six months when stored at refrigerated temperatures.

In one embodiment the composition is an orally administered solution in which the edaravone is completely dissolved in the solvent. The solvent includes propylene glycol or polyethylene glycol, or a combination of the two. A portion of the composition may be water.

In one embodiment, the ratio of the solvent to water may be in the range of about 70/30 to 99/1 and more particularly about 80/20 to 99/1.

In one embodiment, the solution compositions are free of a dispersant. Because the compositions disclosed herein include the edaravone in solution, there is no need to include a dispersant. By being free of a dispersant, the solution composition is free of dispersants such as polyvinyl alcohol, sucrose fatty acid ester, polysorbate, methylcellulose and hypromellose.

In another embodiment, the solution compositions are free of a thickener. Because the compositions disclosed herein include the edaravone in solution, there is no need to include a thickener to keep the particles suspended homogeneously. By being free of a thickener, the solution composition is free of thickening agents such as carmellose sodium, dextrin, tragacanth powder, and xanthan gum and the like.

In another embodiment, the edaravone oral solution does not need to be, and is not, supplied in a polypropylene bag that is further overwrapped with polyvinyl alcohol secondary packaging.

Further the edaravone oral solution does not need to be, and is not, supplied in packaging that includes an oxygen absorber and an oxygen indicator to minimize oxidation.

In one general aspect, the invention relates to a method of treating ALS, the method comprising orally administering a dose of an oral solution of edaravone comprising edaravone in solution, wherein the orally administered dose comprises between 65 and 90 mg of edaravone.

Embodiments of the method may include one or more of the following features. For example, the dose of between 65 and 90 mg of edaravone in solution may be orally administered once per day.

The oral solution may include edaravone and one or both of propylene glycol and polyethylene glycol. The oral solution may include edaravone, purified water, and one or both of propylene glycol and polyethylene glycol. The polyethylene glycol may be selected from a polyethylene glycol characterized by being a liquid at room temperature.

The oral solution may further include sodium bisulfite and L-Cysteine hydrochloride. The oral solution may include edaravone, purified water, propylene glycol, sodium bisulfite and L-cysteine hydrochloride.

The edaravone may be present in the oral solution at a concentration of about 65-90 mg of edaravone per 1-30 mL of the oral solution. The edaravone may be present in the oral solution at a concentration of about 76 mg of edaravone in 5 mL of solution.

The oral solution may have one or both of a geometric mean for Cmax between about 800 ng/mL to about 980 ng/mL and a geometric mean for the AUCi between about 820 ng/mL*hours to about 1,000 ng/mL*hours. The oral solution may have a Tmax of about 0.25 to about 0.50 hours. The oral solution may exhibit an AUCi and/or Cmax that is at least 80% of the mean AUCi and/or Cmax observed for a formulation comprising edaravone in suspension and having a once daily dose of 105 mg. Such a suspension composition may be the RADICAVA ORS® (105 mg/5 mL) oral suspension drug product.

The dose of the oral solution is 76 mg of edaravone and the dose of the oral solution composition is bioequivalent to a dose of an oral suspension of 105 mg of edaravone. Such a suspension composition may be the RADICAVA ORS® (105 mg/5 mL) oral suspension drug product.

In another general aspect, the invention relates to an oral solution of edaravone comprising propylene glycol, wherein the edaravone is dissolved in the propylene glycol and a dose of between 65 and 90 mg of edaravone of the oral solution exhibits an AUCi and/or Cmax that is at least 80% of the mean AUCi and/or Cmax observed for a formulation comprising edaravone in suspension and at a dose of 105 mg of edaravone. Such a composition may be the RADICAVA ORS® (105 mg/5 mL) oral suspension drug product.

Embodiments of the oral solution and its use may include one or more of the following features. For example, the oral solution may comprise edaravone, purified water, propylene glycol and, optionally, polyethylene glycol. The oral solution further includes sodium bisulfite and L-Cysteine hydrochloride monohydrate. The edaravone may be present in the oral solution at a concentration of about 65-90 mg of edaravone per 1-10 mL of the oral solution. Values of any integer between 65 and 90 mg of edaravone may be used with values of any integer between 1 and 30 mL of oral solution. All such combinations are hereby disclosed.

The edaravone may be present in the oral solution at a concentration of about 76 mg of edaravone in about 5 mL of solution. A dose of the oral solution comprises 76 mg of edaravone and the dose of the oral solution composition is bioequivalent to a dose of an oral suspension of 105 mg of edaravone. Such a suspension composition may be the RADICAVA ORS® (105 mg/5 mL) oral suspension drug product.

The oral solution as disclosed or used in the method of treating is chemically stable for six months when stored at either 2-8° C. or 25° C./60% RH.

The oral solution composition consists essentially of, or consists of, purified water, sodium bisulfite, L-Cysteine hydrochloride, propylene glycol and edaravone. The oral solution composition consists essentially of, or consists of, purified water, sodium bisulfite, L-Cysteine hydrochloride, propylene glycol and edaravone and the edaravone is present at a concentration of 76 mg edaravone in 3-10 mL of liquid medium.

The solvent further comprises a water content of equal to or less than about 30% w/w.

In another general aspect, the invention relates to a method treating a condition suitable to being treated by edaravone. The method comprises orally administering a dose of an oral solution of edaravone, the oral solution comprising edaravone in solution. The orally administered dose comprises between 65 and 90 mg of edaravone, the oral solution comprises edaravone and one or both of propylene glycol and polyethylene glycol, and the oral solution exhibits an AUCi and/or Cmax that is at least 80% of the mean AUCi and/or Cmax observed for a formulation comprising edaravone in suspension and having a once daily dose of 105 mg.

Embodiments of the method of treating a condition suitable to being treated by edaravone may include any of the features and aspects disclosed above and herein.

### DESCRIPTION OF THE DRAWINGS

FIG. 1 is a graph showing mean concentration versus time for Treatment A (Test 1), Treatment B (Test 2) and Formulation C (Reference Product).

### DESCRIPTION OF THE INVENTION

Examples of various oral solution compositions of edaravone have been formulated and are listed below. The solution compositions may include inactive ingredients such as one or more stabilizers, sweeteners and/or flavoring agents. Examples of stabilizers include sulfites, bisulfites, and pyrosulfites, cysteines, methionines, polyethylene glycol, polyoxyethylene polyoxypropylene glycol, EDTA, and the like. Examples of sulphites include sodium sulfite (Na₂SO₃), potassium sulfite (K₂SO₃), and calcium sulfite (CaSO₃). Examples of bisulfites include sodium bisulfite (NaHSO₃), potassium bisulfite (KHSO₃), and ammonium bisulfite (NH₄HSO₃). Another example of a suitable sulfite is sodium metabisulfite. Examples of pyrosulfites include sodium pyrosulfite (Na₂S₂O₅) and potassium pyrosulfite (K₂S₂O₅). Further, examples of cysteines include L-cysteine, DL-cysteine, N-acetylcysteine, hydrochlorides thereof, and the like. Stabilizers can be present in the solution compositions at conventional amounts as known in the art. For example, L-cysteine hydrochloride can be present at an amount of about 0.01% (w/v) to about 0.03% (w/v) and sodium bisulfite can be present at an amount of about 0.02% (w/v) to about 0.2% (w/v). In one embodiment, the L-cysteine hydrochloride monohydrate is present at about 0.25 mg/mL (0.025% w/v) and the sodium bisulfite is present at about 0.5 mg/mL (0.05% w/v). L-cysteine hydrochloride should be understood to include all hydrated forms including the monohydrate.

Examples of sweeteners include sugars, artificial sweeteners, and non-sugar sweeteners. Specific examples of sugars include mannitol, sorbitol, xylitol, maltitol, erythritol, sucrose, trehalose, lactose, maltose, glucose, glycerin, and the like. Specific examples of artificial sweeteners include sucralose, aspartame, acesulfame potassium, saccharin, and the like. Specific examples of non-sugar sweeteners include thaumatin, stevia extract, and the like. Sweeteners can be present in the solution compositions at conventional amounts as known in the art and to provide the desired level of sweetness.

Examples of flavors include citrus flavors (such as orange, lemon, and grapefruit), peach, grape, vanilla and berry flavors (such as cherry, strawberry, cranberry, and blueberry). Flavors can be present in the solution compositions at conventional amounts as known in the art and to provide the desired flavor.

The composition is an orally administered solution in which the edaravone is completely dissolved in the solvent. The solvent is selected from combinations of propylene glycol and/or polyethylene glycol, and water. The polyethylene glycol is selected to be a liquid at room temperature. Such polyethylene glycols that meet this requirement include PEG 100 to PEG 800, such as PEG 100, PEG 200, PEG 300, PEG 400, PEG 500, PEG 600, PEG 700 and PEG 800. The ratio of propylene glycol to water may be in the range of about 70/30 to 99/1 and more particularly about 80/20 to 99/1. The ratio of polyethylene glycol to water is similar.

In one embodiment, the solution composition has a pH range of about 3 to about 5. For example, phosphoric acid and sodium hydroxide can be added to adjust the pH.

Example 1

| Materials | g/batch |
|---|---|
| Purified water (liquid medium) | 440 |
| Sodium bisulfite (stabilizer) | 1.1 |
| L-Cysteine hydrochloride monohydrate | 0.55 |
| (stabilizer) | |
| Propylene glycol (liquid medium) | 1700 |
| Edaravone (active ingredient) | 46.2 |
| Propylene glycol (liquid medium) | QS to 2.2 L |

### Preparation Procedure:

1. Added the purified water into a glass beaker
2. Added the L-Cysteine hydrochloride monohydrate and sodium bisulfite into the glass beaker and mixed until dissolved
3. Added the propylene glycol into the glass beaker and mixed well
4. Added the edaravone into the glass beaker and mixed until dissolved
5. QS with propylene glycol to 2.2 L
6. Filled the solution into 60 mL amber glass bottles with child-resistant caps

TABLE 1
Stability of Edaravone Oral Solution of Example
1, 105 mg/5 mL (Lot# RB0472-010)

| | | | | 3M/25° C./ | 3M/40° C./ |
|---|---|---|---|---|---|
| Impurities | RRT | Initial | 3M/2-8° C. | 60% RH | 75% RH |
| Unknown-1 | 1.80 | 0.02 | 0.03 | 0.10 | 0.42 |
| Unknown-2 | 2.06 | — | — | — | 0.18 |
| Total IMP % | — | 0.12 | 0.14 | 0.43 | 1.38 |
| Assay % | — | 99.5 | 99.3 | 98.9 | 98.2 |

The data presented in Table 1 demonstrates that the composition of Example 1 is chemically stable at the conditions reported.

Example 2

| Materials | g/batch |
|---|---|
| Purified water | 520 |
| Sodium bisulfite | 0.52 |
| L-Cysteine hydrochloride monohydrate | 0.26 |
| Propylene glycol | 2000 |
| Edaravone | 54.6 |
| Propylene glycol | QS to 2.6 L |

### Preparation Procedure:

1. Added the purified water into a glass beaker
2. Added the L-Cysteine hydrochloride monohydrate and sodium bisulfite into the glass beaker and mixed until dissolved
3. Added the propylene glycol into the glass beaker and mixed well
4. Added the edaravone into the glass beaker and mixed until dissolved
5. QS with propylene glycol to 2.6 L
6. Filled the solution into 60 mL amber glass bottles with child-resistant caps

### Example 3

Formulations A to E were prepared using the same procedure as in Examples 1 and 2.

### Batch A

| Materials | A (g/batch) |
|---|---|
| Purified water | 16 |
| Sodium bisulfite | 0.04 |
| L-Cysteine hydrochloride monohydrate | 0.02 |
| Ethyl maltol | 0.16 |
| Edaravone | 1.68 |
| Propylene glycol | QS to 80 mL |

| Materials | B (g/batch) |
|---|---|
| Purified water | 16 |
| Sodium bisulfite | 0.04 |
| L-Cysteine hydrochloride monohydrate | 0.02 |
| Butylated Hydroxyanisole (BHA) | 0.08 |
| Edaravone | 1.68 |
| Propylene glycol | QS to 80 mL |

| Materials | C (g/batch) |
|---|---|
| Purified water | 16 |
| Sodium bisulfite | 0.04 |
| L-Cysteine hydrochloride monohydrate | 0.02 |
| Citric acid | 0.4 |
| Edaravone | 1.68 |
| Propylene glycol | QS to 80 mL |

| Materials | D (g/batch) |
|---|---|
| Purified water | 16 |
| Sodium bisulfite | 0.04 |
| L-Cysteine hydrochloride monohydrate | 0.02 |
| Sucralose | 4 |
| Edaravone | 1.68 |
| Propylene glycol | QS to 80 mL |

| Materials | E (g/batch) |
|---|---|
| Purified water | 16 |
| Sodium bisulfite | 0.04 |
| L-Cysteine hydrochloride monohydrate | 0.02 |
| EDTA | 0.08 |
| Edaravone | 1.68 |
| Propylene glycol | QS to 80 mL |

| Materials | g/batch |
|---|---|
| Purified water | 60 |
| Sodium bisulfite | 0.15 |
| L-Cysteine hydrochloride monohydrate | 0.075 |
| Propylene glycol | 150 |
| Sorbitol | 60 |
| Edaravone | 6.3 |
| Propylene glycol | QS to 300 mL |

### Preparation Procedure:

1. Added the purified water into a glass beaker
2. Added the L-Cysteine hydrochloride monohydrate and sodium bisulfite into the glass beaker and mixed until dissolved
3. Added the propylene glycol into the glass beaker and mixed well
4. Added the sorbitol into the glass beaker and mixed until dissolved
5. Added the edaravone into the glass beaker and mixed until dissolved
6. QS with propylene glycol to 300 mL

### Example 5

| Materials | g/batch |
|---|---|
| Purified water | 60 |
| Sodium bisulfite | 0.15 |
| L-Cysteine hydrochloride monohydrate | 0.075 |
| Propylene glycol | 150 |
| Sorbitol | 15 |
| Edaravone | 6.3 |
| Propylene glycol | QS to 300 mL |

### Preparation Procedure:

1. Added the purified water into a glass beaker
2. Added the L-Cysteine hydrochloride monohydrate and sodium bisulfite into the glass beaker and mixed until dissolved
3. Added the propylene glycol into the glass beaker and mixed well
4. Added the sorbitol into the glass beaker and mixed until dissolved
5. Added the edaravone into the glass beaker and mixed until dissolved
6. QS with propylene glycol to 300 mL

### Example 6

### Bioequivalence Study

A bioequivalence study was conducted in the form of a single dose, oral bioequivalence study of two different doses (Treatment A=105 mg in 5 mL and Treatment B=84 mg in 4 mL) of test product edaravone oral solution at 21 mg/mL compared to Radicut® (edaravone) oral suspension, 2.1% (Treatment C=105 mg/5 mL) in healthy adult human subjects under fasting conditions. In summary, in Treatment A 105 mg of edaravone in 5 mL solution was administered, in Treatment B 84 mg of edaravone in 4 mL solution was administered and in Treatment C 105 mg of edaravone in 5 mL was administered.

The objective of the study was to compare and evaluate the oral bioavailability of the two different doses (Dose=105 mg in 5 mL and 84 mg in 4 mL) of the edaravone oral solution (21 mg/mL) (Treatments A and B) to that of Radicut® (edaravone) oral suspension, 2.1% (Dose=105 mg in 5 mL) (Treatment C) in healthy, adult, human subjects under fasting conditions.

Endpoints: The following pharmacokinetic (PK) measurements were calculated to determine bioavailability and bioequivalence of the compositions: Cmax, AUCt, AUCi, Tmax, Kel, AUC_% Extrap_obs and tHalf.

Study Design: An open label, randomized, three-period, three-treatment, six-sequence, crossover, balanced, single dose oral bioequivalence study in healthy adults under fasted condition.

Number of subjects: 18

Housing: The patients were housed for at least 11 hours prior to dosing and through at least 24 hours post dose in each period.

Washout period: The study had a washout period of at least three (3) days.

Administration of investigational products: Subjects received any one of the below mentioned treatments as per the randomization in any of study period:
1) Edaravone Oral solution 21 mg/mL (Dose=105 mg in 5 mL) [Treatment A]
2) Edaravone Oral solution 21 mg/mL (Dose=84 mg in 4 mL) [Treatment B]
3) Radicut® (Edaravone) Oral Suspension, 2.1% (Dose=105 mg in 5 mL) [Treatment C]

Collection of blood samples: In each period, a total 22 venous blood samples (3 mL each) were collected at pre-dose (0.0 hour) and at 0.083, 0.167, 0.25, 0.333, 0.5, 0.667, 0.833, 1.0, 1.25, 1.5, 1.75, 2.0, 3.0, 4.0, 6.0, 8.0, 12.0, 16.0, 18.0, 20.0 and 24.0 hours post dose in labeled K2EDTA* vacutainers through an indwelling cannula placed in the forearm vein/dorsal aspect of hand of the subjects. If required, a sample was alternatively collected through a fresh venipuncture.

The test product was the following edaravone oral solution, 21 mg/mL:

### Example 6 Formulation

| Materials | g/batch |
|---|---|
| Purified water | 1200 |
| L-Cysteine hydrochloride monohydrate | 1.5 |
| Sodium bisulfite | 3.0 |
| Propylene glycol | 4800 |
| Edaravone | 126 |
| Purified water | QS to 6 L |

*Phosphoric acid was added to adjust the pH (3.0-4.5).

The test and reference drugs were administered according to the procedure described above and blood samples taken and analyzed. Tables 2A and 2B report the pharmacokinetic data of the Treatment A Test 1 product (105 mg in 5 mL) and the reference product (Treatment C) Radicut® Oral Suspension. The ratio of the geometric means for Cₘₐₓ for Test 1 Product/Reference Product was 164.89. The ratio of the geometric means for AUCₜ for Test 1 Product/Reference Product was 144.89. The ratio of the AUCᵢ for Test 1 Product/Reference Product was 144.46.

Tables 3A and 3B report the pharmacokinetic data of the Treatment B Test 2 product (84 mg in 4 mL) and the reference product (Treatment C) Radicut® Oral Suspension. The ratio of the geometric means for Cₘₐₓ for Test 2 Product/Reference Product was 115.51. The ratio of the geometric means for AUCₜ for Test 2 Product/Reference Product was 104.30. The ratio of the AUC for Test 2 Product/Reference Product was 104.09.

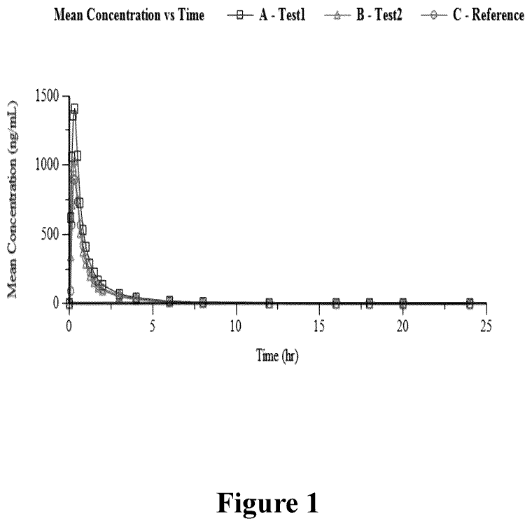

FIG. 1 displays the mean concentration over time for the Treatment A, B and C (RLD) products.

TABLE 2A
Pharmacokinetics of Edaravone Oral Solution, 105 mg in 5 mL, Treatment A

| | | Test-1 Least | Reference Least | Test-1 | Reference | (Test-1/ |
|---|---|---|---|---|---|---|
| | | Square Means | Square Means | Geometric | Geometric | Reference) |
| Parameter | Unit | Ln Data | Ln Data | Means | Mean | Ratio |
| Cmax | (ng/mL) | 7.277 | 6.777 | 1446.923 | 877.518 | 164.89 |
| AUCt | (ng/mL)*(hr) | 7.184 | 6.814 | 1318.721 | 910.130 | 144.89 |
| AUCi | (ng/mL)*(hr) | 7.190 | 6.823 | 1326.617 | 918.323 | 144.46 |

TABLE 2B
Pharmacokinetics of Edaravone Oral Solution,
105 mg in 5 mL, Treatment A

| | | 90% Confidence | Intra- | |
|---|---|---|---|---|
| | | Interval For Test-1 vs | Subject | |
| Parameter | Unit | Reference | CV(%) | A_Power |
| Cmax | (ng/mL) | (129.21%; 210.42%) | 41.892 | 0.0000 |
| AUCt | (ng/mL)*(hr) | (129.93%; 161.59%) | 18.128 | 0.0000 |
| AUCi | (ng/mL)*(hr) | (129.70%; 160.90%) | 17.914 | 0.0000 |

TABLE 3A
Pharmacokinetics of Edaravone Oral Solution, 84 mg in 4 mL, Treatment B

| | | Test-2 Least | Reference Least | Test-2 | Reference | (Test-2/ |
|---|---|---|---|---|---|---|
| | | Square Means | Square Means | Geometric | Geometric | Reference) |
| Parameter | Unit | Ln Data | Ln Data | Means | Mean | Ratio |
| Cmax | (ng/mL) | 6.936 | 6.792 | 1028.732 | 890.581 | 115.51 |
| AUCt | (ng/mL)*(hr) | 6.847 | 6.805 | 940.794 | 902.041 | 104.30 |
| AUCi | (ng/mL)*(hr) | 6.854 | 6.814 | 947.269 | 910.091 | 104.09 |

TABLE 3B
Pharmacokinetics of Edaravone Oral Solution,
84 mg in 4 mL, Treatment B

| | | 90% Confidence | Intra- | |
|---|---|---|---|---|
| | | Interval For Test-2 vs | Subject | |
| Parameter | Unit | Reference | CV(%) | B_Power |
| Cmax | (ng/mL) | (99.05%; 134.71%) | 26.773 | 0.2067 |
| AUCt | (ng/mL)*(hr) | (94.39%; 115.24%) | 17.207 | 0.9147 |
| AUCi | (ng/mL)*(hr) | (94.29%; 114.90%) | 17.032 | 0.9242 |

TABLE 4
Cmax (ng/mL)

| | 105 mg in 5 mL | 84 mg in 4 mL | RLD 105 mg |
|---|---|---|---|
| Value | dose | dose | in 5 mL |
| No. (patients) | 17 | 18 | 18 |
| Mean | 1566.753 | 1126.028 | 1029.111 |
| SD | 639.640 | 470.333 | 544.213 |
| CV % | 40.826 | 41.769 | 52.882 |
| Min | 459.900 | 346.600 | 284.300 |
| Median | 1526.000 | 1102.000 | 895.900 |
| Maximum | 2617.000 | 2148.000 | 1896.000 |
| Geometric Mean | 1419.015 | 1028.732 | 890.581 |

TABLE 5
AUCt (ng/mL)*(hr)

| | 105 mg in | 84 mg in | RLD 105 mg |
|---|---|---|---|
| Value | 5 mL dose | 4 mL dose | in 5 mL |
| No. (patients) | 17 | 18 | 18 |
| Mean | 138.577 | 978.673 | 982.999 |
| SD | 463.642 | 278.595 | 430.195 |
| CV % | 33.414 | 28.467 | 43.763 |
| Min | 521.404 | 509.430 | 484.077 |
| Median | 1456.297 | 993.287 | 917.615 |
| Maximum | 2460.874 | 1712.785 | 2044.332 |
| Geometric Mean | 1305.681 | 940.797 | 902.037 |

TABLE 6
AUCi (ng/mL)*(hr)

| | 105 mg in | 84 mg in | RLD 105 mg |
|---|---|---|---|
| Value | 5 mL dose | 4 mL dose | in 5 mL |
| No. (patients) | 17 | 18 | 18 |
| Mean | 1395.303 | 985.111 | 991.006 |
| SD | 465.088 | 279.453 | 432.542 |
| CV % | 33.332 | 28.368 | 43.647 |
| Min | 527.399 | 515.053 | 492.314 |
| Median | 1466.300 | 999.158 | 923.462 |
| Maximum | 2473.366 | 1720.709 | 2065.355 |
| Geometric Mean | 1313.528 | 947.264 | 910.084 |

TABLE 7
Tmax (hr)

| | 105 mg in | 84 mg in | RLD 105 mg |
|---|---|---|---|
| Value | 5 mL dose | 4 mL dose | in 5 mL |
| No. (patients) | 17 | 18 | 18 |
| Mean | 0.294 | 0.361 | 0.344 |
| SD | 0.094 | 0.107 | 0.105 |
| CV % | 31.850 | 29.634 | 30.529 |
| Min | 0.083 | 0.250 | 0.250 |
| Median | 0.333 | 0.333 | 0.333 |
| Maximum | 0.500 | 0.500 | 0.500 |
| Geometric Mean | 0.275 | 0.374 | 0.330 |

TABLE 8
Kel (1/hr)

| | 105 mg in | 84 mg in | RLD 105 mg |
|---|---|---|---|
| Value | 5 mL dose | 4 mL dose | in 5 mL |
| No. (patients) | 17 | 18 | 18 |
| Mean | 0.178 | 0.181 | 0.180 |
| SD | 0.028 | 0.036 | 0.050 |
| CV % | 15.642 | 19.707 | 27.710 |
| Min | 0.125 | 0.111 | 0.084 |
| Median | 0.179 | 0.187 | 0.186 |
| Maximum | 0.240 | 0.238 | 0.248 |
| Geometric Mean | 0.176 | 0.177 | 0.172 |

TABLE 9
Thalf (hr)

| | 105 mg in | 84 mg in | RLD 105 mg |
|---|---|---|---|
| Value | 5 mL dose | 4 mL dose | in 5 mL |
| No. (patients) | 17 | 18 | 18 |
| Mean | 3.991 | 4.001 | 4.288 |
| SD | 0.664 | 0.941 | 1.703 |
| CV % | 16.636 | 23.529 | 39.729 |
| Min | 2.892 | 2.916 | 2.797 |
| Median | 3.875 | 3.700 | 3.726 |
| Maximum | 5.536 | 6.217 | 8.242 |
| Geometric Mean | 3.942 | 3.909 | 4.040 |

The Cmax, AUCₜ and AUCᵢ data above, and in particular the ratio of these values for the oral solution products compared to corresponding values for the RLD product show that the 105 mg dose was above the desired ratios but the 84 mg dose was within the desired range of ratios but on the higher end. A summary of the relevant data from FIG. 1 is presented in Table 10. Of note, the geometric mean for the Cmax of the RLD was about 890 ng/mL and the geometric mean for the AUCᵢ of the RLD was about 910 ng/mL*hours to. The Tmax was about 0.333 hours. An objective of the compositions of the invention is for the oral solution to be within about 80% to about 125% of these values for Cmax, AUCt and AUCᵢ.

TABLE 10
Test/RLD PK Ratios

| | (Test-1/Reference) | (Test-2/Reference) |
|---|---|---|
| Parameter | Ratio | Ratio |
| Cₘₐₓ | 164.89 | 115.51 |
| AUCₜ | 144.89 | 104.30 |
| AUCᵢ | 144.46 | 104.09 |

### Chemical Stability Evaluation

A pilot batch of edaravone oral solution (21 mg/mL) according to the formulation of Example 6 was filled into amber glass bottles equipped with child-resistant closures. The samples were subjected to stability studies under the following storage conditions: 2-8° C. (refrigerated), 25° C./60% RH (long-term) (LT), 30° C./65% RH (intermediate) (IN), and 40° C./75% RH (accelerated) (AC). Tables 11 and 12 report the test results of the stability samples. Chemical stability requires impurities to be less than 0.200 at a particular time period to be considered chemically stable for that time period under the specified storage conditions.

TABLE 11
Stability Evaluation, One and Two Months

| | | | 1 M/ | 1 M/ | 1 M/ | 1 M/ | 2 M/ | 2 M/ | 2 M/ | 2 M/ |
|---|---|---|---|---|---|---|---|---|---|---|
| Sample | | Initial | 2-8° C. | LT | IN | AC | 2-8° C. | LT | IN | AC |
| Impurity | Highest | BLOQ | BLOQ | BLOQ | BLOQ | BLOQ | BLOQ | BLOQ | BLOQ | 0.22% |
| | unknown | | | | | | | | | |
| | impurity | | | | | | | | | |
| | Total | BLOQ | BLOQ | BLOQ | BLOQ | BLOQ | BLOQ | BLOQ | BLOQ | 0.33% |
| | IMP % | | | | | | | | | |

| | | | | | | | | | |
|---|---|---|---|---|---|---|---|---|---|
| Assay % | 97.8% | 99.6% | 99.8% | 99.9% | 99.4% | 97.3% | 97.4% | 96.8% | 96.6% |
| pH | 3.67 | 3.72 | 3.64 | 3.57 | 3.52 | 3.63 | 3.61 | 3.47 | 3.40 |

BLOQ = below level of quantification

TABLE 12
Stability Evaluation, Three and Six Months

| | | 3 M/ | 3 M/ | 3 M/ | 3 M/ | 6 M/ | 6 M/ | 6 M/ | 6 M/ |
|---|---|---|---|---|---|---|---|---|---|
| Sample | | 2-8° C. | LT | IN | AC | 2-8° C. | LT | IN | AC |
| Impurity | Highest | BLOQ | BLOQ | 0.11% | 0.32% | BLOQ | 0.11% | 0.23% | 2.03% |
| | unknown | | | | | | | | |
| | impurity | | | | | | | | |
| | Total | BLOQ | BLOQ | 0.11% | 0.46% | BLOQ | 0.21% | 0.35% | 3.06% |
| | IMP % | | | | | | | | |

| | | | | | | | | |
|---|---|---|---|---|---|---|---|---|
| Assay % | 100.5% | 100.0% | 99.7% | 99.1% | 98.3% | 98.0% | 97.1% | 94.3% |
| pH | 3.64 | 3.46 | 3.38 | 3.36 | 3.54 | 3.43 | 3.39 | 3.42 |

BLOQ = below level of quantification

Based on the differences between the pharmacokinetic data for the oral solution product given at 105 mg in 5 mL and 84 mg in 4 mL, as described above, the inventors determined that the dose should be reduced to ensure bioequivalence to the reference product. Using the data above the inventors applied linear regression techniques to determine that the oral dose of an edaravone solution should be in the range of about 69-88 mg edaravone in a single dose of an oral solution to fall within the range of 80-125% of the RLD product for Cmax; and in the range of about 35-126 mg edaravone in a single dose of an oral solution to fall within the range of 80-125% of the RLD product for AUCi. Therefore, to ensure that both the Cmax and AUCi are within the range of 80-125% of the RLD product, the edaravone oral solution should be administered at a dose of about 69-88 mg of edaravone. In other embodiments the dose is in a range of about 65-90 mg of edaravone in oral solution, a range of about 66-89 mg of edaravone in oral solution, a range of about 67-88 mg of edaravone in oral solution, a range of about 68-87 mg of edaravone in oral solution, a range of about 69-86 mg of edaravone in oral solution, a range of about 70-85 mg of edaravone in oral solution, a range of about 71-84 mg of edaravone in oral solution, a range of about 72-83 mg of edaravone in oral solution, a range of about 73-82 mg of edaravone in oral solution, a range of about 74-81 mg of edaravone in oral solution, a range of about 75-80 mg of edaravone in oral solution, a range of about 76-79 mg of edaravone in oral solution, or a range of about 77-78 mg of edaravone in oral solution, that should be administered to be within the range of 80-125% of the RLD product for both Cmax and AUCi.

In another aspect, the concentration of the drug in the solution can be varied so long as the single dose is in the range of about 65-90 mg edaravone administered to the patient. Therefor an edaravone drug solution of about 65 mg/l mL and an edaravone drug solution of about 65 mg/10 mL would provide the desired dose. Similarly, an edaravone drug solution of about 87 mg/l mL and an edaravone drug solution of about 87 mg/10 mL would provide the desired dose. Similarly, an edaravone drug solution of about 76 mg/l mL to 76 mg/10 mL would provide the desired dose.

The concentration can be varied based on factors such as taste, convenience, stability, and ease of administration.

Accordingly, a flavored edaravone oral solution composition has the following composition and is intended to be administered at a dose of about 76 mg (5 mL) to a patient.

### Example 7

Proposed formula: Edaravone oral solution, 76 mg/5 mL

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Sucralose | 5 |
| Purified water | QS to 1 mL |

Phosphoric acid is added to adjust the pH (3.0-4.5).

In one aspect, the composition consists of water and propylene glycol as the liquid medium, one or more stabilizers, and the active ingredient:

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Purified water | QS to 1 mL |

Phosphoric acid is added to adjust the pH (3.0-4.5).

In another aspect, the composition consists of water and propylene glycol as the liquid medium, one or more stabilizers, the active ingredient and one or both of a flavor and a sweetener.

In another aspect, the composition consists of the following edaravone oral solution, 76 mg/5 mL:

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Sweetener, e.g., sucralose | 5 |
| Purified water | QS to 1 mL |

Phosphoric acid is added to adjust the pH (3.0-4.5).

In one aspect, the composition consists essentially of water and propylene glycol as the liquid medium, one or more stabilizers, and the active ingredient. A novel aspect of the composition is the ability to provide an oral solution of edaravone that is stable at room temperature for three or more months. Another novel aspect of the composition is the ability to provide an oral solution of edaravone that is stable at room temperature for three or more months using water and propylene glycol as the liquid medium, one or more stabilizers and the edaravone active ingredient dissolved in the liquid medium. In another aspect, the composition consists essentially of purified water (liquid medium), sodium bisulfite (stabilizer), L-Cysteine hydrochloride monohydrate (stabilizer), propylene glycol (liquid medium), and edaravone (active ingredient). Such a composition can also include a flavor or sweetener although they are not essential components of the composition.

In another aspect, the composition consists essentially of the following composition:

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Purified water | QS to 1 mL |

Phosphoric acid is added to adjust the pH (3.0-4.5).

In another aspect, the composition consists essentially of purified water, L-Cysteine hydrochloride monohydrate, sodium bisulfite, propylene glycol, edaravone, and a sweetener, e.g., sucralose.

In another aspect, the composition consists essentially of the following edaravone oral solution, 76 mg/5 mL:

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Sweetener, e.g., sucralose | 5 |
| Purified water | QS to 1 mL |

Phosphoric acid is added to adjust the pH (3.0-4.5).

In another aspect, the composition consists of water and propylene glycol as the liquid medium, one or more stabilizers, the active ingredient and one or both of a flavor and a sweetener.

In another aspect, the composition consists of the following edaravone oral solution, 76 mg/5 mL:

| Materials | mg/mL |
|---|---|
| Purified water | 200 |
| L-Cysteine hydrochloride monohydrate | 0.25 |
| Sodium bisulfite | 0.5 |
| Propylene glycol | 800 |
| Edaravone | 15.2 |
| Sucralose | 5 |
| Purified water | QS to 1 mL |

Phosphoric acid is added to adjust the pH (3.0-4.5).

### Example 8. Additional Formulation Examples

Batch A. Edaravone Oral Solution (76 mg/5 mL)

| Materials | g/batch |
|---|---|
| Purified water | 500 |
| Sodium bisulfite | 1.25 |
| L-Cysteine hydrochloride monohydrate | 0.25 |
| Propylene glycol | 2,000 |
| Edaravone | 38 |
| Sucralose | 12.5 |
| Purified water | QS to 2.5 L |

*Phosphoric acid was added to adjust the pH (3.0-5.0).

Batch B. Edaravone Oral Solution (105 mg/5 mL)

| Materials | g/batch |
|---|---|
| Purified water | 320 |
| Sodium bisulfite | 0.8 |
| L-Cysteine hydrochloride monohydrate | 0.4 |
| Propylene glycol | 1,280 |
| Edaravone | 33.6 |
| Citric acid | 12.5 |
| Purified water | QS to 1.6 L |

Batch C. Edaravone Oral Solution (105 mg/5 mL)

| Materials | g/batch |
|---|---|
| Purified water | 440 |
| Sodium bisulfite | 1.1 |
| L-Cysteine hydrochloride monohydrate | 0.55 |
| Propylene glycol | 1,600 |
| Edaravone | 46.2 |
| Disodium hydrogen phosphate dihydrate | 3.43 |
| Propylene glycol | QS to 2.2 L |

*Phosphoric acid was added to adjust the pH (3.0-5.0).

It is understood that the concentration of edaravone in the liquid medium can be modified and still fall within the scope of the invention. For example, in some aspects of the invention, the compositions disclosed herein can be modified to increase the amount of liquid medium in a dose to be 2 mL, 3 mL, 4 mL, 5 mL, 6 mL, 7 mL, 8 mL, 9 mL, etc. For example, the formulation can be modified to have a concentration of about 15.2 mg edaravone/1-10 mL liquid medium. The composition is administered to ensure that about 76 mg of edaravone is administered to the patient in a single dose.

The formulations disclosed herein provide unexpected results compared to both the RADICAVA® injection product and the RADICAVA ORS® oral suspension product. With respect to the RADICAVA® injection product, the formulations disclosed herein have unexpectedly improved storage stability. As the prescribing information for RADICAVA® indicates, the product must be packaged with an inner polypropylene bag, an overwrapping of polyvinyl alcohol and an oxygen scavenger and indicator in the overwrapping to minimize oxidation. Even with such protection from oxygen, once the overwrap packaging is opened, the product must be used within 24 hours. In contrast, the compositions of the invention are stable for six months when stored at 2-8° C. and 25° C. and 60% RH; at 30° C. and 65% RH for up to 6 months; and at 40° C. and 75% RH for up to two months.

With respect to the RADICAVA ORS® suspension product, the formulations disclosed herein unexpectedly provide reduced dosages of edaravone compared to the suspension product while being bioequivalent to the suspension product. In particular, a composition of about 84 mg of edaravone in solution was found to be bioequivalent to a composition of 105 mg of edaravone in suspension. As set out above, the solution composition of about 84 mg of edaravone had a Cmax within 80% and 125% of the Cmax of the RADICAVA ORS® 105 mg suspension product. In particular, the ratio of the Cmax of the 84 mg edaravone solution to the Cmax of the 105 mg edaravone suspension product was found to be between 0.80 and 1.25. Further, the solution composition of about 84 mg of edaravone also had a AUCi within 80% and 125% of the AUCi of the RADICAVA ORS® 105 mg suspension product. The ratio of the AUCi of the 84 mg edaravone solution to the AUCi of the 105 mg edaravone suspension product was found to be between 0.80 and 1.25. It was unexpected that a 20% reduction in the dosage could result merely by forming a solution rather than a suspension.

The bioequivalence between the edaravone solution composition of the invention and the RADICAVA ORS® suspension composition based on Cmax and/or AUCi can be expected for a range of edaravone of about 65 mg to about 90 mg, in particular about 75 mg to about 77 mg, about 76 mg, based on the above linear regression analysis.

The formulations disclosed herein are suitable for treating ALS or another condition for which edaravone is or may be suitable. In a method of treating ALS or other condition, the edaravone solution is administered once daily to a patient. The edaravone composition should be administered after the patient has fasted, such as for about 2-8 hours. The patient should continue to fast after administration, such as for about one hour.

It should be understood that the descriptions of the compositions disclosed herein are to be interpreted to permit the composition to comprise the listed ingredients and amounts, to consist essentially of the listed ingredients and amounts, and/or to consist of the listed ingredients and amounts. Therefore the claims are intended to encompass compositions that comprise, consist essentially of or consist of the recited ingredients and amounts.

What is claimed:

1. A method of treating ALS, the method comprising orally administering a dose of an oral solution of edaravone comprising edaravone in solution, wherein the orally administered dose comprises between 65 and 90 mg of edaravone.

2. The method of claim 1, wherein the dose of between 65 and 90 mg of edaravone in solution is orally administered once per day.

3. The method of claim 1, wherein the oral solution comprises edaravone and one or both of propylene glycol and polyethylene glycol.

4. The method of claim 1, wherein the oral solution comprises edaravone, purified water, and one or both of propylene glycol and polyethylene glycol.

5. The method of claim 3, wherein the oral solution further comprises sodium bisulfite and L-Cysteine hydrochloride.

6. The method of claim 5, wherein the oral solution comprises edaravone, purified water, propylene glycol, sodium bisulfite and L-cysteine hydrochloride.

7. The method of claim 1, wherein the edaravone is present in the oral solution at a concentration of about 65-90 mg of edaravone per 1-30 mL of the oral solution.

8. The method of claim 7, wherein the edaravone is present in the oral solution at a concentration of about 76 mg of edaravone in 5 mL of the oral solution.

9. The method of claim 1, wherein the oral solution has a geometric mean for Cmax between about 800 ng/mL to about 980 ng/mL and has a geometric mean for the AUCi between about 820 ng/mL*hours to about 1,000 ng/mL*hours.

10. The method of claim 1, wherein the oral solution has a Tmax of about 0.25 to about 0.50 hours.

11. The method of claim 1, wherein the oral solution exhibits an AUCi and/or Cmax that is at least 80% of the mean AUCi and/or Cmax observed for a formulation comprising edaravone in suspension and having a once daily dose of 105 mg.

12. The method of claim 1, wherein the dose of the oral solution comprises 76 mg of edaravone and the dose of the oral solution composition is bioequivalent to a dose of an oral suspension of 105 mg of edaravone.

13. An oral solution of edaravone comprising propylene glycol, wherein the edaravone is dissolved in the propylene glycol and a dose of between 65 and 90 mg of edaravone of the oral solution exhibits an AUCi and/or Cmax that is at least 80% of the mean AUCi and/or Cmax observed for a formulation comprising edaravone in suspension and at a dose of 105 mg of edaravone.

14. The oral solution of claim 13, wherein the oral solution comprises edaravone, purified water, propylene glycol and, optionally, polyethylene glycol.

15. The oral solution of claim 13, wherein the oral solution further comprises sodium bisulfite and L-Cysteine hydrochloride monohydrate.

16. The oral solution of claim 13, wherein the edaravone is present in the oral solution at a concentration of about 65-90 mg of edaravone per 1-30 mL of the oral solution.

17. The oral solution of claim 13, wherein the edaravone is present in the oral solution at a concentration of about 76 mg of edaravone in about 5 mL of solution.

18. The oral solution of claim 17, wherein a dose of the oral solution comprises 76 mg of edaravone and the dose of the oral solution composition is bioequivalent to a dose of an oral suspension of 105 mg of edaravone.

19. The oral solution of claim 13, wherein the oral solution is chemically stable for at least six months when stored at either 2-8° C. or 25° C./60% RH.

20. The oral solution of claim 13, wherein the solution composition consists essentially of purified water, sodium bisulfite, L-Cysteine hydrochloride, propylene glycol and edaravone.

21. The oral solution of claim 13, wherein the solution composition consists essentially of purified water, sodium bisulfite, L-Cysteine hydrochloride, propylene glycol and edaravone and the edaravone is present at a concentration of 76 mg edaravone in 3-10 mL of liquid medium.

22. The oral solution of claim 13, where the solvent further comprises a water content of equal to or less than about 30% w/w.

23. A method treating a condition suitable to being treated by edaravone, the method comprising orally administering a dose of an oral solution of edaravone, the oral solution comprising edaravone in solution, wherein
the orally administered dose comprises between 65 and 90 mg of edaravone,
the oral solution comprises edaravone and one or both of propylene glycol and polyethylene glycol, and
a dose of the oral solution containing about 76 mg of edaravone exhibits an AUCi and/or Cmax that is at least 80% of the mean AUCi and/or Cmax, respectively, observed for a formulation comprising edaravone in suspension and having a once daily dose of 105 mg.
